(19) United States
(12) Patent Application Publication
(10) Pub. No.: US 2007/0189965 A1
(43) Pub. Date: Aug. 16, 2007

(54) Copper-complex isonitrile positron emission tomography (pet) imaging agent and method

(76) Inventors: John H. Kuperus, Tampa, FL (US); Robert G. Mckenzie, Tampa, FL (US); Brooke Schumm, Ellicott City, MD (US)

Correspondence Address: BROOKE SCHUMM III, ONE NORTH CHARLES STREET, SUITE 2450, BALTIMORE, MD, 21201, US

(21) Appl. No.: 11/570,774

(22) PCT Filed: Jun. 17, 2005
(86) PCT No.: PCT/US05/21561

§ 371 Date: Dec. 17, 2006

Related U.S. Application Data
(60) Provisional application No. 60/580,455, filed on Jun. 17, 2004.
(60) Provisional application No. 60/608,080, filed on Sep. 8, 2004.
(60) Provisional application No. 60/522,619, filed on Oct. 20, 2004.
(60) Provisional application No. 60/522,940, filed on Nov. 23, 2004.
(60) Provisional application No. 60/595,249, filed on Jun. 17, 2005.

(30) Foreign Application Priority Data
Oct. 22, 2004 (US) 10904099

(51) Int. Cl. A61K 51/00

(52) U.S. Cl. 424/1.65

(57) ABSTRACT

A novel method is set out of preparation of Copper isonitrile as a radioactive diagnostic radiopharmaceutical in a stable, shippable, lyophilized from by an apparatus designed to rapidly flash freeze and dehydrate a radiopharmaceutical composition to minimize auto radiolysis. The Copper radiopharmaceutical is can be reconstituted and administered to a patient. The method proposes rapid cooling and removal of ambient vapor, and then ultra cold removal when the potential of explosive liquid oxygen is eliminated. The radioactive diagnostic radiopharmaceutical requires no further cold or refrigerated storage, including with respect to shipping, subsequent to stabilization. The preferred composition can be reconstituted “on site” by the addition of a suitable diluent to bring the radiopharmaceutical complex into solution at a desired concentration.

### CONTINUATION DATA

This is a continuation-in-part of provisional application No. 60/580,455 entitled Stabilized and Lyophilized Radiopharmaceutical Agents filed on Jun. 17, 2004 and a provisional application No. 60/608,060 of that name filed on Sep. 8, 2004, and a provisional application No. 60/522,619 filed on Oct. 20, 2004, and related to a co-pending U.S. utility application Ser. No. 10/904,099 entitled Stabilized and Lyophilized Radiopharmaceutical Agents, and U.S. Provisional 60/522,940 filed on Nov. 22, 2004, and U.S. provisional No. 60/595,249 filed on Jun. 17, 2005 of the same name as this invention, which are adopted by reference.

### FIELD OF INVENTION

This invention relates to tagging of a particular lipophilic ligand isonitrile, with or without additional substitutents upon it, and a Cu-64 positron-emitting isotope for positron emission tomography (PET) scanning, and the stabilization and lyophilization of such tagged ligands.

The inventors propose a novel composition and method using a new combination, and a stabilized composition and method for creating that stabilized use, of Cu-64 positron emitters for administration to patients and imaging of those patients in PET scanners, and production of a stabilized lyophilized Cu-64 isonitrile product.

### BACKGROUND

Isonitrile has been used in conjunction with a Technetium tag for some time for purposes of gamma camera imaging. Jones, U.S. Pat. No. 4,452,774, Jun. 5, 1984, and J. Nucl. Med. Vol. 23, No. 5, P16-P17, June, 1982. Unfortunately, despite the widespread use of Technetium for imaging, Technetium is not suitable for use with positron emission tomography (PET) scanners. PET scanners can produce sharp images, but thus far no method or suggestion of a suitable radionuclide with a suitable ligand and a half-life that enables overnight delivery or storage has been made for PET imaging. Existing isotopes for PET imaging are normally F-18 and N-13 which have half lives of less than two hours.

The general mechanism of technetium imaging is believed to be that the resultant complex from the Jones art is a complex that has a +1 charge. That complex acts a mimic to a potassium ion which has a +1 charge. Because the heart has a potassium pump, meaning a chemical mechanism that causes potassium to be transported into the heart muscle, a successful mimic could be similarly transported. The concept behind the Jones art was that if the mimic had a radioactive tag and if it was lipophilic, the lipophilic mimic would associate and concentrate in conjunction with fatty tissue in dysfunctional parts of the heart muscle, and upon imaging of the radioactive portions of the heart, the extent of heart disease and function could be ascertained. At the time of the Jones patent applications, PET machines were relatively rare, and there was no necessity of developing a PET compatible imaging product.

As time has moved on, the theory behind technetium imaging has been confirmed. However, in part because there was no need, and in part because it just has not been done, no successful PET imaging using isonitrile has been developed. Isonitrile has a lipophilic affinity, i.e., an affinity for fatty tissue. Complicating the issue is that copper is not itself transportable by a cationic pump unless complexed with isonitrile. In order to effect the transport, it is important that the entire complex of isonitrile have an ionic charge of +1, rather than such charge being isolated in the Copper ion itself. Also, Cu-64 has not been readily available for commercial use on an economic basis. Because Cu-64 has only a 12.7 hour half life, Cu-64 is difficult to work with in conjunction with isonitrile which is very volatile.

A copper isonitrile⁺¹ complex which the inventors propose has characteristics that mimic K⁺¹ and therefore it is transported as a mimic into the heart muscle—the areas with poor circulation have less uptake of potassium and the copper isonitrile and upon imaging with PET scanner, can identify areas of poor circulation by the lack of uptake contrasted with areas of better circulation.

Carpenter et al, U.S. Pat. No. 4,894,445, Jan. 16, 1990, identified preparation of Tc isonitrile made by substitution of Tc for non-radioactive Cu on an isonitrile complex. A later patent, Carpenter et al, U.S. Pat. No. 5,324,824, contemplated replacing non-radioactive copper isonitrile with a radioactive adduct. This generates a potential gamma camera Imaging compound, including for cardiac imaging. The result compound(s) identified in the Carpenter art referenced are not suitable for PET imaging. A positron-emitting radionuclide with sufficient emission levels would need to be selected.

The background to the stabilized lyophilization procedure is as follows.

While the efficacy of radioactive diagnostic and therapeutic agents is established, it is also well known that the emitted radiation can cause substantial chemical damage or destabilization to various components in radiopharmaceutical preparations, referred to as autoradiolysis. Emitted radiation causes the generation of free radicals in water solutions, which free radicals are generally peroxides and superoxides. Such free radicals can precipitate proteins present in the preparations, and can cause chemical damage to other substances present in the preparations. Free radicals are molecules with unbonded electrons that often result because the emissions from the radioactive element can damage molecules by knocking apart water molecules forming hydroxyl radicals and hydrogen radicals, leaving an element or compound with a shell of charged electrons which seek to bond with other molecules and atoms and destabilize or change those molecules and atoms. The degradation and destabilization of proteins and other components caused by the radiation is especially problematic in aqueous preparations. Under the present art, the radiolysis causes the aqueous stored ligand and radioactive isotope bonded to the ligand to degenerate and destroys the complex which renders it useless for imaging because the biological characteristics that localize the complex to a tissue are gone. The degradation or destabilization lowers or destroys the effectiveness of radiopharmaceutical preparations, and has posed a serious problem in the art. Wahl, et al, Journal of Nuclear Medicine, Vol 31, Issue 1 84-89, discuss the fact that freezing radiolabeled antibodies at −70 degrees C. stabilizes the molecule for an indefinite period but 80 to 90% of the immunoreactivity is lost in as little as 24 hours when stored at 4 degrees C.

Secondly, and much less known, the danger of too much cooling of the atmosphere surrounding a radiopharmaceutical is that oxygen molecules are liquefied, generating a potent oxidizer, which oxidizer is also deleterious to radiopharmaceutical preparations. Staged cooling and evacuation as proposed in this invention is the preferred mode to deal with this problem. Placement under an inert gas can also reduce but may not completely eliminate this problem.

If the ligands are permitted to reside with the radioactive elements for an extended period, particularly in an aqueous (water-based) solution, the radiolysis is increased. Thus, any process to reduce the compounds to dried form has to be rapid and yield predictable result. Further, to avoid the higher concentrations and protect the ligands, presently the radiopharmaceutical solution is diluted, but that in itself only slows the drying time and complicates the problem and increases the unpredictability of the non-radioisotope portion of the radiopharmaceutical because of radiolysis. Heating the radiopharmaceutical in solution to accelerate the drying and removal of water has the undesirable effect of potentially damaging the ligand since chemical activity normally increases upon heating or injection of energy and therefore the effects of radiolysis are also increased during this prolonged drying period with heating. Most proteins are badly damaged upon heating. Certain ligands, such as isonitrile, simply evaporate and disappear upon heating. Further, minimization of localized heating at an atomic scale is important to preserve both the small quantities needed and to yield a specific concentration of desired product.

Reichel, U.S. Pat. Nos. 2,066,302, Dec. 29, 1936, and 2,085,392, Jun. 29, 1937, made a serious effort to preserve biologically active substances, though the patents do not suggest they are useful for chemical agents. Those Reichel patents proposed rapid freezing of the fresh liquid substance, and then “removal of water from the solid frozen material without melting or softening thereof by the application of a high vacuum and regulated warming of the material without melting or softening.” See, for example, U.S. Pat. No. 2,085,392, p. 1, col. 1, line 11. Reichel characterized his '302 art as relating to containers for storing the frozen materials. In the '302 patent, he mentioned that “In order to increase the rate of evaporation of the water in the frozen material, where this material is at too low a temperature, the charged chamber may be warmed with warm circulating air or by immersing it in a warm liquid, but the heat applied should not be sufficient to melt or even soften the frozen charge.” U.S. Pat. No. 2,066,302, p. 2, col. 1, line 25. No suggestion of the method and results of this invention are given in the Reichel art, particularly the necessity of avoiding and dealing with the problem of liquid oxygen. Reichel's proposed method of accelerating freezing, Reichel '302, p. 3, col. 1, line 71, was: “the rate of evaporation of the ice from the frozen material may be increased by warming the container, where it is at too low a temperature, by circulating warm air over it or by immersing it in a warm liquid, but the heat applied should never be sufficient to melt or even soften the frozen charge.” Further, the Reichel art is not designed for radiopharmaceutical applications and is not designed for overcoming the problems of radiolysis. By contrast to the Reichel art, the method of the present invention proposes further cooling of evacuated gas by a secondary condenser simultaneous to further vacuum evacuation to increase the rate of evaporation from the frozen material. By contrast to the Reichel art, this patent proposes a method of dealing with liquid oxygen by a process with stages, or less preferably, use of inert gas to avoid liquid oxygen problems.

Wolfangel, U.S. Pat. No. 5,219,556, Jun. 15, 1993, entitled stabilized therapeutic radiopharmaceutical complexes, expressed his concern as follows: “The isotopes which are most useable with this process are determined by practical considerations. Again, Tc-99m would be a poor candidate for use since its six-hour half-life makes lyophilization impractical, as the lyophilization step itself generally takes about 24 hours to perform.”

Facially, the '556 invention seemed to identify a useful process and resulting composition, but the lyophilization step in '556 invention, as the application stated, took about 24 hours. The '556 invention stated: “The lyophilization is carried out by pre-freezing the product, and then subjecting the frozen product to a high vacuum to effect essentially complete removal of water through the process of sublimation. The resultant pellet contains the complex in an anhydrous form which generally can be stored indefinitely, with practical consideration being given to the half-life of the radionuclide. The intended period of storage for radiopharmaceutical products is thus practically limited by the half-life of the radionuclides. In the case of Re-186, for example, the desired period of storage would range from 7 to about 30 days. Thus, this pellet can be shipped to the end users of the product and reconstituted with a diluent at the time of administration to the patient with very little effort on the part of the health care professional and/or nuclear pharmacist.”

Because the procedures in '556 did not rapidly lyophilize the product, and contemplated a 24 hour period for lyophilization, the claims of '556 invention were necessarily limited to utilization of a “therapeutic amount of an alpha- or beta-emitting radionuclide.” Wolfangel had observed that compounds with a half-life of at least 12 hours are preferred. However, the concentrations required of a short half-life compound are too high to be stored and used with Wolfangel's process because the radionuclide would damage the remainder of the radiopharmaceutical. By contrast, the use of Tc-99m, which also emits gamma rays, with a half-life of only six hours, or the use of other similarly short-lived radioisotopes, becomes impractical.

Wolfangel '556 proposed in his example 1 to first lyophilize certain compounds, add the radionuclide complex, sparge with gas, seal the vial and then heat it. Unfortunately, the heating to 11 degree C. renders the procedure useless in conjunction with most proteins or peptides, and many commonly used complexes. Further, the proposal was to use 1 ml of sodium perrhenate Re-186 containing 1 mg of rhenium, with water added to produce 3 ml. The quantities contemplated were substantial and exposed the workers to substantial amounts of radiation. In example 3, it was proposed that the complex be frozen to −30 degree C. or colder and then apply a vacuum, but it was proposed to apply shelf heat at 6 degree per hour until a product temperature of 30 degree C. was reached, at which time the temperature would be held for two hours. That would require 12 hours. The procedure suffered from the infirmity of not quickly removing water and therefore not preventing radiolysis of the water and not preventing the generation of free radicals which damage the complexes. The second example 2 followed the first, but used smaller quantities, and proposed heating. Example 3 proposed heating to 85 degree C. for 30 minutes which would destroy most proteins and thereafter freezing and lyophilizing the sealed vials.

In contrast to the Wolfangel '556 invention which stated: “the lyophilization step itself generally takes about 24 hours to perform,” the present invention proposes to produce a stable radiopharmaceutical complex by a lyophilization process which “freeze-dries” the complex in five hours or less, normally 2-4 hours, and then requires no further refrigeration.

The inventors propose a novel method of PET imaging using a new combination, and a stabilized cationic composition of Cu-64 isonitrile produced by a process, and a method for creating that stabilized composition of a Cu-64 positron emitter for administration to patients and imaging in PET scanners.

### OBJECTIVES OF THE INVENTION

An object of the invention is to describe a new method of PET cardiovascular imaging using a Cu-64 isonitrile complex, particularly for cardiovascular and cerebrovascular imaging.

An object of the invention is to stabilize and lyophilize the Cu-64 isonitrile so it can be stored or shipped by accelerating the removal of water to minimize the peroxidation-related effects of radiolysis because of the accelerated removal of water. That accelerated removal which facilitates stabilization and predictability of concentration of a ligand or non-radioactive portion of a radiopharmaceutical because of reduced radiolysis.

An object of the invention is to use the minimization of peroxidation-related effects to improve the preservation of the Cu-64 isonitrile complex.

An object of the invention is to use small quantities of Cu-64 isonitrile at concentrations which enable accelerated lyophilization, longer predictable storage and overnight shipment, and increase worker safety. Corollary to this objective is the elimination of need for cold storage and refrigeration.

An object of the invention is to use vials with an expanded surface area, extremely cold temperatures and very low level pressures without heating in combination to accelerate lyophilization.

An object of the invention is to use a two stage system to accelerate lyophilization by not only lowering vacuum pressure, but also, after initial removal of oxidizing agents, to extract vapor more rapidly by supercooling gas being evacuated.

### DESCRIPTION OF INVENTION

The inventors have developed a novel product and process of making the product to accomplish a PET scannable mimic of the potassium pump into the heart, which is particularly suitable for PET cardiac imaging.

The solution is non-obvious over existing art.

For instance, it is not possible in any commercially meaningful way to merely substitute off the Technetium in a Technetium t-butylisonitrile complex with a Cu-64 ion.

The most expedient way to manufacture Tc t-butylisonitrile is to mix zinc isonitrile with Tc-glucoheptonate and stannous chloride and heat it. The result is a water soluble compound Tc complex useful for cardiac imaging.

Unfortunately, such a procedure does not work with Cu-64. Zinc isonitrile mixed with Cu⁶⁴-glucoheptonate, or mixed additionally with stannous chloride, will not yield a useful product for PET imaging.

An expected reaction might be to use Cu⁶⁴X where X is a halogen anion and combine it with a standard substitution reaction of a Y—(CN)ₓ compound where Y is a suitable cation. An example might be:
Cu⁶⁴Br₂+Zn(CN)₂−Cu⁶⁴(CN)₂+ZnCl₂.

This too does not yield a pharmaceutically acceptable product.

The inventors have conceived a process that results in a novel composition that yields a radioactive tag that can be imaged by a PET scanner, and is a potassium mimic suitable for cardiac imaging.

The inventors propose to take a quantity, for instance of 10 mg-100 mg MIBI (also referred to as 1-isocyano-2-methoxy-2-methyl-propane or referred to as 2-methoxy isobutyl isonitrile) available from ABX, Advanced Biochemical Compounds at Heinrich-glaesser-strasse 10-14, 01454 Raderberg, Germany. That quantity of isonitrile would be put in a suspension in 100 mL of trichloromethane in, preferably an appropriate inert atmosphere, including under a non-reactive gas such as argon or nitrogen. That quantity selected would be the necessary amount in the suspension to have an excess of isonitrile compared to the ultimate amount of Cu-64 to be complexed. The desired amount of Copper-64 ion in the form of CuZ, where Z is a halogen in dry or dehydrated form would be added and the mixture would be warmed up to not more than 79° C. This results in the formation of isonitrile complexed around the Cu64 bearing a charge of +1 (referred to as Cu(CN)ₐ⁺¹ or Cu(CN)ₐ) in solution with a Z⁻ anion. The solution would be put in a filter resistant to organic solvents, smaller than approximately 0.22 μm, in order to filter out the unreacted CuZ. A clear solution would result and be evaporated to about 10% of the original 100 mL volume. Then a quantity of about 50% of the original volume of the suspension (in this example 50 mL) of diethyl ether would be added. The Cu(CN)ₐZ would precipitate out and be filtered again to collect the precipitate. The precipitate would then be dissolved in water or normal saline dissociating on dissolution to yield a solution of positively charged Cu-64 isonitrile complex.

The complex CU⁶⁴(CN)ₐZ, may have any number of pharmaceutically acceptable organic radicals bonded to the CN portion of the complex, provided the CN is adjacent to the Cu64. The formula is then Cu⁶⁴(CN)ₐR⁺¹Z, where R is an organic radical known to a reasonably skilled practitioner in the field, including organic radicals referenced in Jones et al, U.S. Pat. Nos. 4,452,774, 4,707,544, 4,826,961, and 4,872,561 and the above referenced Carpenter art and art cited in those patents.

The preferred mode of the invention is to prepare an imaging quantity of copper isonitrile of approximately 10 mCi (millicuries) from the saline solution and administer it to a patient. However, several doses could be combined in a vial, and doses can range from 10mCi to 500mCi. For greater amounts of millicurie doses in that range, or for longer storage over many half-lives, the inventors propose that the solution be stabilized according to the following procedure.

The preferred mode of the composition produced by stabilization and lyophilization in this invention is with respect to Cu-64 isonitrile radionuclides. The method of treatment by PET scanning using Cu-64 isonitrile radionuclides, in particularly for cardiovascular imaging is set out. The following illustrates the compositions and processes of this invention, but is not meant to limit the scope of the invention in any way.

Cu64(CN)ₐ⁺¹ is prepared as above. The concentration is increased so that ultimately one-half milliliter or less will equal one dose. For example the usual dose of Cu64(CN)a⁺¹ for a typical patient would be 10 mCi (millicuries). Because the half life of Cu-64 is about 12.7 hours, in order to allow for normal radioactive decay in shipment so that one dose is 10 mCi (millicuries) upon administration, 20 mCi would be generated in solution and drawn into a 10 mL vial on the prior day anticipating overnight shipment.

In order to achieve the objects of this invention, and in contrast to the 24 hour lyophilization period set out in Wolfangel '556, this invention proposes to use the following apparatus. First, the vial will be stoppered with a sterile lyophilization stopper. For this invention, a lyophilization stopper is a stopper which permits flow of vapor. The preferred stopper is a “three-legged” stopper which has grooves to permit equalization of pressure between the interior of the vial and the ambient atmosphere in which the vial(s) is (are) present. A typical three legged stopper that is suitable for the invention is a three-legged n-butyl rubber lyophilization stopper 224100-202 manufactured by Wheaton Pharmaceutical of Wheaton, Ill. The vials will be placed in a tray in the shape of a standard round baking pan with a perimeter wall of about 1 inch. The vials are flat bottomed and are set in a tray which is shaped like a standard round baking pan with a perimeter wall of about 1 inch. The tray will be placed into a stoppering frame. The tray in the stoppering frame will be set in a chamber in the lyophilization apparatus. The stoppering frame will be place on an inner tube placed on top of it that can be inflated before the vacuum is broken in the chamber to force the stoppers against a flat surface farther into the vials after dehydration in order to seal the vials. Other mechanical devices are available to seal the vials.

The chamber, according to the procedure set forth below, will ultimately be used to not only receive the tray in the stoppering frame but also is designed to be sealed to enable a vacuum and other steps in the procedure to be undertaken.

The chamber is composed of a base which is a preferably a flat sheet of Lexan or acrylic material because of their strength. If the base is Lexan, it would be preferably about ½″ thick and if acrylic, about 1 inch thick. The base is 14×14 inches and is larger than the open-ended acrylic cylinder of 12 inches in diameter and 18 inches high which is contemplated to be placed upon the flat sheet. The cylinder should be made of at least ¼ inch thick material. The ends of the acrylic material that are exposed are covered with a gas-tight seal usually of rubber or silicone. The purpose of the seal is to enable the cylinder to be set on the base and form a gas tight seal by the weight of the cylinder upon the seal. The chamber has a sealable port to accommodate a connection from the exterior of the chamber through a hose to the inner tube on the stoppering frame.

The lid of the chamber is also either Lexan or acrylic of sufficient strength to withstand the vacuum which will be placed upon it. If the lid is Lexan, it would be preferably about ½″ thick and if acrylic, about 1 inch thick. The lid has a gas valve on the lid which enables entry of gas to flow into the chamber.

Another access port which consists of a one-inch rubber stopper is located centrally on the lid which will be used in case the gas valve fails and will enable a needle to be inserted to relieve the vacuum in the chamber. The rubber stopper also has situated in it an electrical connector to enable a wire connecting a thermistor probe, which will be on at least one of the vials, to be connected through the stopper to an outside monitoring device. A thermistor is the easiest among many means to measure temperature.

The bottom plate has a two inch hole in it which has an adapter connected to it to enable a hose to be connected to the base of the chamber in order to evacuate gas from the chamber which chamber will eventually be sealed. The evacuation hose is of sufficient strength to withstand the contemplated vacuum. The end of the hose which is not attached to the base of the chamber is attached to a secondary condenser which will not be initially activated. The secondary condenser will ultimately be maintained at a much colder temperature than the initially activated primary condenser. The secondary condenser is a stainless tube of approximately one inch diameter. When the secondary condenser is activated, that tube in the secondary condenser will be surrounded by supercool liquid Nitrogen that will be maintained at a temperature of around −196 degree C. This is much colder than the freezing agent suggested in the Reichel art of liquid air, dry ice (solidified carbon dioxide), and such cold temperature in this invention would interfere with the proposed acceleration of evaporation by warming that Reichel suggested.

A hose is connected from the secondary condenser to the primary condenser.

The primary condenser is a stainless steel pot which has a bottom with an aperture and an adapter connected to that aperture to which adapter is attached a drain hose which can be sealed. The stainless steel pot of the primary condenser is made of Y4 inch stainless steel, and can be sealed, and is approximately 8 liters in volume and capable of withstanding the vacuum.

The primary condenser is surrounded by a standard refrigeration system capable of lowering the temperature to at least −40 C.

At the commencement of the lyophilization procedure, the primary condenser will have had its temperature lowered to −40 degrees C.

The condensing system is heavily insulated.

A hose runs from the top or side of the stainless steel pot of the primary condenser to the vacuum pump.

A vacuum pump capable of producing a vacuum of at least 10⁻⁴ Torr, and preferably 10⁻⁶ Torr would be used to evacuate the chamber. An appropriate vacuum pump is model RV-12 available from BOCEdwards, an international company, through the internet at edcom.bocedwards.com.

In order to achieve the composition contemplated in this invention, the primary condensing coil is readied at or below −40 deg. C. Promptly after mixing the radiopharmaceutical composition, the vial containing the radiopharmaceutical composition, in the preferred mode a vial of 10 mL or less, preferably containing 1 mL or less of aqueous Cu⁶⁴(CN)a⁺¹Z⁻¹, is stoppered with the lyophilization stopper, with the lyophilization stopper in a position to permit passage of vapour. The vial and stopper will be fully sealed at the end of the process.

The vial(s) is (are) placed into the tray and a sufficient amount of liquid nitrogen is poured onto the tray in order to flash freeze the vials by the heat transfer from the aqueous Cu64(CN)ₐ⁺¹Z⁻¹ through the sides of the vial. Because of the small quantity which is used and the high surface area of the vial, the freezing occurs virtually instantaneously. The tray is placed into a stoppering frame in the chamber with the inner tube connected and installed so that at the end of the procedure, before the vacuum is broken, the port to the inner tube can be opened and the tube will inflate and force the stoppers fully into the vials in order to seal them.

As the liquid nitrogen evaporates off, a thermistor on one of the vials is connected to the electrical connector on the rubber stopper which connects to an outside temperature monitoring device. The liquid nitrogen is allowed to evaporate, all the while maintaining the temperature of the vial at or below −10 degrees C.

The top of the chamber is installed and forms a seal with the cylindrical side of the chamber. After evaporation of the liquid nitrogen, the gas valve on top of the chamber is closed, and the rubber stopper is installed.

After the tray containing the flash-frozen vials is placed into the chamber, and the chamber has been sealed, the vacuum pump is turned on. A vacuum pressure is first felt in the primary condenser and any vapor in the chamber begins to flow out through the secondary condenser and freezes in the primary condenser which is kept at a temperature above the boiling point of oxygen, meaning preferably kept at about −40 degrees C. When the vacuum pump gauge shows 10⁻³ Torr, usually after about 20 minutes, liquid Nitrogen at −196 degrees C. is allowed to flow through the secondary condenser and cool the stainless steel tube contained in the secondary condenser through which gas evacuated from the chamber is flowing. The very cold liquid Nitrogen in the secondary compressor is used to increase the temperature difference between the secondary condenser and the vial contents to accelerate the lyophilization. The secondary condenser is placed in series with the primary condenser and the evacuated chamber containing the tray of vials. The secondary condenser takes over as the larger and colder heat sink to capture the vaporized water. The vacuum pump continues to operate until the pressure is about 10⁻⁶ Torr. The process is complete in under five hours, by contrast to the 12 hours referenced by Wolfangel. The accelerated process in this invention compared to Wolfangel enables use of higher concentrations of radionuclide without destruction of the ligand.

In summary, the method begins by flash-freezing the radiopharmaceutical composition in the vial(s). The vials are lyophilization stoppered, and placed in the lyophilization tray which is placed in the apparatus. The apparatus is sealed. The pressure is lowered to at least 10⁻² Torr and preferably 10⁻³ Torr, and the temperature in the evacuation tube is cooled by the primary condenser to −40 degrees C. Counterintuitively to the Reichel art, U.S. Pat. Nos. 2,066,302, Dec. 29, 1936, and 2,085,392, Jun. 29, 1937, in order to accelerate the dehydration and reduce autoradiolysis, instead of warming the composition as Reichel proposes, while keeping it below freezing, the inventors here further cool and further depressurize the composition to −170 degree C., and to 10⁻⁶ Torr.

Because the acrylic chamber has no refrigeration, the temperature of the vial and the vial contents after initial cooling, will ultimately tend to rise above 0 degrees C. after all of the water is removed. This signals the completion of the cycle. The thermistor probe connected through the rubber stopper to the outside monitoring device enables the monitoring of the vial temperature. The vials would then be sealed in partial pressure of pharmaceutically inert gas that is fully dehydrated or “dry,” meaning gas that is non-reactive with the pharmaceutical composition, the gas preferably being argon or nitrogen. In contrast with the Reichel art which proposed leaving the vials evacuated, this invention contemplates sealing with pharmaceutically inert gas because without the partial pressure of that gas, with the much lower pressures of 10⁻⁶ Torr vacuum contemplated in this invention, the vials would be difficult to open without contaminating the radiopharmaceutical in the vial. An inner tube will have been placed in the chamber to be inflated to force the stoppers into the vial to seal them. An auxiliary cylinder of gas that is chemically inert relative to the lyophilized radionuclide is used to gradually inflate the inner tube through the valve to force the stoppers into the vials. The vacuum is broken. The vial stoppers further secured with an aluminum seal. At the end of the process upon warming, the water which was frozen and subsequently melted will be drained from the primary condenser.

The vials are ready to be shipped with predictable half lives for the radionuclide and a stabilized ligand in powdered form. The radioactive diagnostic radiopharmaceutical in this invention requires no further cold or refrigerated storage, including with respect to shipping, subsequent to stabilization.

If it is desired to accelerate the lyophilization process, inert gas may be temporarily admitted through the gas valve into the chamber to displace any oxygen and enable the secondary condenser to be turned on sooner. The displacement is necessary to prevent accumulation of liquid oxygen in the secondary condenser. In the ordinary procedure, if the secondary condenser is activated before the 10⁻³ Torr level is reached, there is a risk of collecting liquid oxygen which is potentially explosive.

The secondary condenser is in series with the primary condenser, and could be located subsequent to the primary condenser in the evacuation and condensing system.

The speed of the lyophilization process is positively influenced by the lowering of the vapor pressure external to the material being dried. Secondly, the speed is positively influenced by the greater temperature difference between product being cooled and the temperature of the condenser where the water is being collected.

The lyophilized radiopharmaceutical composition is reconstituted “on site” for administration to patients by the addition of a suitable diluent to bring the radiopharmaceutical complex into solution at the time of administration to the patient.

For administration, the Cu64(CN)ₐ⁺¹Z⁻¹ in the vial must be reconstituted. Because of the minute quantity of material, the vial of radionuclide complex, in the preferred mode the Cu64(CN)ₐ⁺¹Z⁻¹ will appear empty. The Cu64(CN)ₐ⁺¹ ligand is stable for several days because of the absence of water which is the primary substance from which free radicals are generated by radioactive particle collisions with water molecules. The particle rays are being emitted by the radionuclide, that is the Cu-64. The health care provider would add up to 2 ml. of sterile normal saline. The desired dose would be withdrawn and measured in a dose calibrator of a type manufactured by Capintec of Montville, N.J. If the glass vial is measured in the dose calibrator, the person measuring the dose must recognize that the glass vial will decrease the apparent activity. Upon calibration of the desired dose, the Cu64(CN)ₐ⁺¹Z⁻¹ now re-dissolved in the solution is promptly administered to the patient.

The advantages are that the flash freezing and lowering of vapor pressure result in quick formation and evaporation or sublimation (evaporation from ice to water vapour (a gas)) of water from the Cu64(CN)ₐ⁺¹Z⁻¹. Cu64(CN)ₐ⁺¹Z⁻¹ need not be shipped frozen in dry ice nor need it be shipped for overnight delivery. Shipping in dry ice over a weekend is generally not commercially practical. The Cu64(CN)ₐ⁺¹Z⁻¹ can be shipped over the weekend and be used on Monday while simply maintaining it at room temperature or below.

The micro quantities involved for radionuclide complexes such as Cu64(CN)ₐ⁺¹Z⁻¹ substantially reduce the exposure of production workers and health care providers because minute quantities are involved.

It is generally preferable to apply the flash-freezing first because application of the reduced pressure may cause the solution to boil out of the vial.

The stable, lyophilized, radiopharmaceutical composition is reconstituted with suitable diluent at the time of intended use, thus greatly simplifying the on-site preparation process.

In order to establish the advantages of the novel process and resulting composition, a series of tests were run utilizing meta iodo benzyl guanidine (MIBG) in which the radionuclide I-131 was the iodine in the MlBG.

The MIBG was prepared as follows: eight vials were prepared of MIBG in solution with a radioactive concentration of MIBG of 1 mCi per vial. The MIBG in six of those vials were then stabilized and lyophilized according to the process described in this invention. One vial was frozen and maintained at a temperature of −10 degrees, and another vial was simply refrigerated at approximately 5 degrees.

Six vials were prepared according to the process in this invention in order to enable several to be reconstituted from the lyophilized state and their activity tallied.

The radioactive concentration of MIBG per vial was 1 mCi per vial.

The results showing the percent of iodine remaining bound to the MIBG are set forth in table 1. One each of the vials was reconstituted after 24, 48, 72 and 168 hours respectively.

| | | | | | 168 hours |
|---|---|---|---|---|---|
| | 0 hours | 24 hours | 48 hours | 72 hours | (1 wk.) |
| Lyophilized | 96.3% | 97% | 96.6% | 96.2% | 95.9% |
| and | | | | | |
| stabilized | | | | | |
| per | | | | | |
| invention | | | | | |
| stored at | | | | | |
| room temp. | | | | | |
| Frozen −10° | 96.3% | 94% | 91% | 84% | 72% |
| Refrigerated | 96.3% | 92% | 85% | 77% | 55% |
| ˜+5° | | | | | |

In sum, the radiolysis damage was virtually eliminated from the composition stabilized and lyophilized under this invention while, as the prior art suggests, MIBG that was not so stabilized and lyophilized per this invention deteriorated sharply in activity.

As another example, I-131 Hippuran was prepared. The I-131 Hippuran was prepared as follows: 9 vials were prepared of I-131 Hippuran in solution with a radioactive concentration of MIBG of 1 mCi per vial. Each vial had 4 cc. The I-131 in seven of those vials was then stabilized and lyophilized according to the process described in this invention. One vial was frozen and maintained at a temperature of −10 degrees, and another vial was maintained room temperature. Room temperature was selected because Hippuran is thought to be stable at room temperature even in conjunction with a radioisotope.

The results showing the percent of Hippuran remaining bound to the 1-123 are set forth in table 2. One each of the vials was reconstituted after 24, 48, 72 and 168 hours respectively.

TABLE 2

| | 0 hours | 24 hrs | 48 hrs | 72 hrs | 96 hrs | 120 hrs |
|---|---|---|---|---|---|---|
| Lyophilized | 98% | 98.4% | 98.6% | 98% | 98.4% | 98.5% |
| and stabilized | | | | | | |
| per invention | | | | | | |
| stored at | | | | | | |
| room temp. | | | | | | |
| Frozen −10° | 98% | 97.8% | 97% | 94% | 92.5% | 91 |
| Room Temp. | 98% | 96% | 95% | 94.5% | 92% | 90% |

In sum, the radiolysis damage was virtually eliminated from the composition stabilized and lyophilized under this invention.

If one desires to ship product, maintaining a product reliably frozen even at −10 degrees is difficult and expensive as a practical matter; this invention makes such shipment practical over the techniques of the prior art. One reference has suggested that storage at −70° C. can limit autoradiolysis damage, but even in that article, the percent free iodine, e.g. unbonded iodine, rose from what appears to be 1.6% to 4.3% in 24 hours. Wahl, Inhibition of Autoradiolysis of Radiolabeled monoclonal Antibodies by Cryopreservation, 31(1) J. Nucl. Med. 84-89 (January 1990). Conversely, putting those results in a form analogous to Table 1, the percentage of free iodine in the Wahl article commenced at 98.4% and fell in 24 hours to 95.7% in Wahl's Table 1. The contrast between that fall in bonded iodine in 24 hours of some 3.7% in the Wahl reference versus a fall of 0.4% during a week for the composition stabilized and lyophilized per this invention illustrates the sharp advantage of the present method and resulting composition. In addition, it is not practical in real-world conditions to replenish the cooling fluid to maintain −70° C. much less to ship it cost-effectively.

The radiopharmaceutical composition which results from the method of this invention may be further purified after reconstitution, if desired. One method of purification is described in EP 250966, noted above. Other methods are known to those skilled in the art.

The radiopharmaceutical composition can include other components, if desired. Useful additional components include chemical stabilizers, lyophilization aids and microbial preservatives. Such chemical stabilizers include ascorbic acid, gentisic acid, reductic acid, para-amino benzoic acid, and erythorbic acid among others. In some cases, these agents are beneficial in protecting the oxidation state of the radionuclide by preferential reaction with oxygen or by direct effect. The term lyophilization aids includes those substances known to facilitate good lyophilization of the product. These aids are used to provide bulk and stability to the dried pellet and include lactose, dextrose, albumin, gelatin, sodium chloride, mannitol, dextran and pharmaceutically-acceptable carriers, among others. Antimicrobial preservatives inhibit the growth of or kill microbial contaminants which are accidentally added to the product during preparation. The term antimicrobial preservatives includes methylparaben, propylparaben and sodium benzoate. These components generally are added to the composition after the complex has been formed between the ligand and the radionuclide but prior to lyophilization. Bacteriastatic agents, for example, methyl and propyl-paraben may be added. Also contemplated are the addition of solubilizing agents such as polyethylene glycol to enhance the solubility of fatty

What is claimed is:

1. A method of preparing a stable rapidly lyophilized radiopharmaceutical composition of a copper radioisotope with ligand having an isonitrile-base substitutent for diagnostic or therapeutic purposes that needs no refrigeration upon completion of the method and that increases the predictability of the integrity of the radiopharmaceutical composition by reducing radiolysis damage, comprising the following steps:
evacuating a sealable chamber containing a flash frozen amount of said radiopharmaceutical composition having a copper radioisotope with a ligand having an isonitrile-base substitutent in at least one lyophilization-stoppered but as yet unsealed vial, said flash frozen amount being frozen preferably in an ultracold freezing shelf or in liquefied gas, preferably nitrogen, said evacuating of said sealable chamber occurring by a vacuum pump connected by an evacuation tube passing through a primary condenser and a secondary condenser down to a pressure sufficient to eliminate the explosive potential of liquid oxygen while maintaining the temperature of said primary condenser above the boiling point of oxygen;
accelerating the removal of water from said sealable chamber by activating said secondary condenser to reduce said evacuation tube temperature, preferably down to a temperature above the boiling point of nitrogen of approximately −196 Celsius, thereby reducing more rapidly the presence of water molecules, including radiolysis degenerated water molecules, and reducing attendant free radical damage to said radiopharmaceutical composition, and increasing the predictability of the integrity of the radiopharmaceutical composition; and
upon completion of the desired removal of water, restoring the ambient pressure in the sealable chamber to close to atmospheric pressure with a pharmaceutically inert gas, and upon such restoration of ambient pressure, sealing the said at least one vial in order to preclude entry of external fluid.

2. The method according to claim 1, further comprising:
said radiopharmaceutical composition utilizing MIBI.

3. The method according to claim 1, further comprising:
said ligand being a ligand selected from the group of isonitrile ligands used with Tc cardiac imaging.

4. The method according to claim 1, further comprising:
said copper radioisotope being Cu-64.

5. The method according to claim 1, further comprising:
said evacuating said sealable chamber occurring at a primary condenser temperature of approximately −40 degrees C. until said pressure sufficient to eliminate the explosive potential of liquid oxygen has reached approximately 10⁻² Torr.

6. The method according to claim 5, further comprising:
said radiopharmaceutical composition utilizing sestamibi (MIBI).

7. The method according to claim 5, further comprising:
said ligand being a ligand selected from the group of isonitrile ligands used with Tc cardiac imaging.

8. The method according to claim 5, further comprising:
said copper radioisotope being Cu-64.

9. A method of treatment of a patient with stabilized rapidly lyophilized radiopharmaceutical composition of a copper radioisotope with ligand having an isonitrile-base substitutent for diagnostic or therapeutic purposes that needs no refrigeration pending administration, with increased predictability of the integrity of the radiopharmaceutical composition by reducing radiolysis, comprising the following steps:
reconstituting a radiopharmaceutical composition having at least one radionuclide which composition has been prepared by
a) evacuating a sealable chamber containing a flash frozen amount of said radiopharmaceutical composition having a copper radioisotope with a ligand having an isonitrile-base substitutent in a lyophilization-stoppered but as yet unsealed vial, said flash frozen amount being frozen preferably in an ultracold freezing shelf or in liquefied gas, preferably nitrogen, said evacuating of said sealable chamber occurring by an evacuation tube passing through a primary condenser for cooling and a secondary condenser for cooling down to a pressure sufficient to eliminate the explosive potential of liquid oxygen while maintaining the temperature of said primary condenser above the boiling point of oxygen;
b) accelerating the removal of water from said sealable chamber by activating said second condenser to reduce said evacuation tube temperature to a temperature above the boiling point of nitrogen of approximately −196 Celsius thereby reducing more rapidly the presence of water molecules, including radiolysis degenerated water molecules, and reducing attendant free radical damage to said radiopharmaceutical composition, and increasing the predictability of the integrity of the radiopharmaceutical composition; and
c) upon completion of the desired removal of water, restoring the ambient pressure in the sealable chamber to close to atmospheric pressure with a pharmaceutically inert gas, and upon such restoration of ambient pressure, sealing the vials in order to preclude entry of external fluid, said radiopharmaceutical having a ligand selected from the group of isonitrile ligands useable in cardiac imaging; and
administering said composition having been reconstituted to said patient.

10. The method according to claim 9, further comprising:
said radiopharmaceutical composition utilizing MIBI.

11. The method according to claim 9, further comprising:
said ligand being a ligand selected from the group of isonitrile ligands used with Tc cardiac imaging.

12. The method according to claim 9, further comprising:
said copper radioisotope being Cu-64.

13. The method according to claim 9, further comprising:
said evacuating said sealable chamber occurring at a primary condenser temperature of approximately −40 degrees C. until said pressure sufficient to eliminate the explosive potential of liquid oxygen has reached approximately 10⁻² Torr.

14. The method according to claim 13, further comprising:
said radiopharmaceutical composition utilizing MIBI.

15. The method according to claim 13, further comprising:
said ligand being a ligand selected from the group of isonitrile ligands used with Tc cardiac imaging.

16. The method according to claim 13, further comprising:
said copper radioisotope being Cu-64.

17. A method of preparing a stable rapidly lyophilized radiopharmaceutical composition of a copper radioisotope with a ligand having an isonitrile-base substitutent for diagnostic or therapeutic purposes, comprising the following steps:
a) evacuating a sealable chamber containing a flash frozen amount of said radiopharmaceutical composition having a copper radioisotope with a ligand having an isonitrile-base substitutent in at least one lyophilization-stoppered but as yet unsealed vial, said flash frozen amount being frozen preferably in an ultracold freezing shelf or in liquefied gas, preferably nitrogen, said evacuating of said sealable chamber occurring by a vacuum pump connected by or through an evacuation tube passing through a primary condenser and a secondary condenser down to a pressure sufficient to eliminate the explosive potential of liquid oxygen while maintaining the temperature of said primary condenser above the boiling point of oxygen;
b) accelerating the removal of water from said sealable chamber by activating said secondary condenser to reduce said evacuation tube temperature, preferably down to a temperature above the boiling point of nitrogen of approximately −270° C.; and
c) upon completion of the desired removal of water, restoring the ambient pressure in the sealable chamber to close to atmospheric pressure with a pharmaceutically inert gas, and upon such restoration of ambient pressure, sealing the said at least one vial in order to preclude entry of external fluid.

18. The method according to claim 17, wherein said evacuating said sealable chamber is occurring at a primary condenser temperature of approximately −40° C. until said pressure sufficient to eliminate the explosive potential of liquid oxygen has reached approximately 10⁻² Torr.

19. The method according to claim 17 or 18, wherein said ligand in said radiopharmaceutical composition is selected from the group of isonitrile ligands used with Technetium cardiac imaging.

20. The method according to claim 17 or 18, wherein said isonitrile ligand is MIBI.

21. (canceled)

22. (canceled)

23. The method according to claims 17-20 further comprising:
Administering to a patient said radiopharmaceutical composition, said composition comprising an imaging agent having a selective affinity for the hepatobiliary system and/or for the cardiac system and/or the cerebral system and/or the skeletal system for imaging a patient.

24. The method according to claims 17-20, further comprising:
Administering to a patient said radiopharmaceutical composition, said composition comprising an imaging agent used for prostate imaging and/or for pulmonary imaging while imaging a patient.
